(19) United States
(12) Patent Application Publication
(10) Pub. No.: US 2026/0364177 A1
(43) Pub. Date: Nov. 19, 2026

(54) Composition with Polar and Non-Polar Phase

(71) Applicant: PM-International AG, Schengen (LU)

(72) Inventors: Rolf Sorg, Schengen (LU); Markus Herrmann, Konz (DE); Axel Archalmbault, Volmerange-les Mines (FR); Rudi Wajda, Mannheim (DE); Horst Meinhardt, Herschbach (DE)

(21) Appl. No.: 19/515,678

(22) PCT Filed: Aug. 26, 2024
(86) PCT No.: PCT/EP2024/073803

§ 371 Date: Mar. 2, 2026

(30) Foreign Application Priority Data
Aug. 29, 2023 (DE) 10 2023 123 167.7

(51) Int. Cl. A61K 47/46; A61K 9/10; A61K 31/122; A61K 31/355; A61K 31/455; A61K 31/525; A61K 47/10; A61K 47/24; A61K 47/44

(52) U.S. Cl. CPC A61K 47/46; A61K 9/10; A61K 31/122; A61K 31/355; A61K 31/455; A61K 31/525; A61K 47/10; A61K 47/24; A61K 47/44

(57) ABSTRACT

The invention relates to a composition, wherein the composition comprises a polar phase and a non-polar phase, wherein
the composition contains a lipophilic extract from ginger (Zingiber officinale) and at least one other lipophilic ingredient,
the polar phase contains at least 20% by weight of polyol, at least 3% by weight and less than 20% by weight of water, and at least one hydrophilic ingredient, based on the total weight of the polar phase, and
the composition contains 0.5 g to 10 g of lecithin per 100 g of the composition.

The invention relates to a composition comprising a polar phase and a nonpolar phase.

Two-phase systems, i.e., systems with a polar phase and a nonpolar phase, are an important dosage form, e.g., for dietary supplements or cosmetic preparations.

WO 2019/233552 A1 describes the need to provide two-phase systems that are droppable and transparent and exhibit good physical, chemical, and microbiological stability despite the use of naturally occurring ingredients. Describes are also emulsions, i.e., complex two-phase systems consisting of two immiscible phases, one of which is stably distributed in droplet form in an outer phase. Three types of stability determine the long-term quality and storage properties of emulsions and similar two-phase systems: physical stability, chemical stability, and microbiological stability.

Physical stability describes the ability of a two-phase system to maintain the fine and uniform distribution of the dispersed phase achieved during emulsification by mechanical forces over a longer period of time. It can be improved, for example, by using surfactants or emulsifiers, equalizing the density of the two phases, or increasing the viscosity of the outer phase using so-called quasi-emulsifiers. Surfactants are divided into anionic, cationic, amphoteric, and nonionic surfactants, with nonionic surfactants offering several advantages: they do not form ions in aqueous solutions, react neutrally, are not affected by electrolytes, and are more indifferent to chemical influences.

In the dietary and cosmetic sectors transparent or at least translucent two-phase systems are particularly desirable. In some cases, this can be achieved by means of an inner phase with droplet sizes in the nanometer range. Such droplet sizes can be achieved, for example, by micelles, whose formation can be mediated by an emulsifier and a co-emulsifier. Ethoxylated surfactants or synthetic surfactants with a high HLB value are often used as emulsifiers, and alcohols are used as co-emulsifiers. Emulsifying oils often requires a considerable amount of emulsifier and co-emulsifier. However, synthetic emulsifiers are rejected by some people. In addition, alcohols such as ethanol and propanol are cytotoxic and must be avoided, especially in oral preparations for children, alcoholics, pregnant women, etc.

In addition to the physical stability of a two-phase system, chemical and microbiological stability must also be ensured. Chemical stability is a particular problem in two-phase systems, as autoxidation and hydrolytic processes in oils and fats are promoted in the presence of water. Furthermore, air and thus oxygen are often incorporated during the manufacture of two-phase systems, which also promotes oxidation. This is problematic in that, for example, oxidized fatty acids are no longer only unavailable for physiological purposes, but oxidation also produces products that are harmful to health. In practice, it is usually avoided to incorporate hydrophilic ingredients into two-phase systems in addition to solvents, as this often has an additional negative effect on chemical stability.

As water-containing systems, two-phase systems are good breeding grounds for microorganisms. Therefore, preservatives are usually added to such two-phase systems. However, the surfactants required for physical stability can impair the effectiveness of the preservatives. Preservatives also partially transfer to the lipophilic phase. To ensure that the concentration of preservatives in the aqueous phase is high enough to achieve preservation, such two-phase systems have a relatively high preservative content. However, certain preservatives, such as p-hydroxybenzoic acid esters, can cause intolerances and allergies.

Many people want products based on compatible and naturally occurring ingredients or excipients, although this is often technically difficult to achieve. Two-phase systems such as emulsions are particularly complex systems in which ingredients cannot be mixed together arbitrarily. Instead, a mixture must be developed that meets the needs of the user and the high technical requirements for physical, chemical, and microbiological stability.

The present invention is based on the task of providing a further improved two-phase system that enables improved nutrient transport. At the same time, the two-phase system should be as digestible as possible, not trigger any allergies, and be positively received by humans both psychologically and physiologically. The two-phase system should not contain any monohydric alcohols, in particular no ethanol or propanol. Furthermore, the two-phase system should be physically, chemically, and microbiologically stable, although only naturally occurring ingredients and additives should be used as far as possible. Synthetic emulsifiers and preservatives should be avoided.

According to the invention, this task is solved by a composition according to claim 1.

The invention is based on the idea of specifying a composition comprising a polar phase and a non-polar phase, wherein the composition contains a lipophilic extract from ginger (Zingiber officinale) and at least one further lipophilic ingredient, the polar phase contains at least 20 wt. % polyol, at least 3 wt. % and less than 20 wt. % of water and a hydrophilic ingredient, and the composition contains 0.5 g to 10 g of lecithin per 100 g of the composition.

In addition to lecithin and lipophilic ginger extract and in addition to water and polyol in the polar phase, the composition contains at least one hydrophilic (synonym: polar) ingredient in the polar phase and at least one further lipophilic (synonym: nonpolar) ingredient in the nonpolar (synonym: lipophilic) phase. The composition allows hydrophilic and hydrophobic ingredients to be applied simultaneously in a stable dosage form and these ingredients to be absorbed particularly efficiently by the human body. Due to the presence of a polar phase and a non-polar phase, the composition according to the invention can implement a nutrient transport concept (NTC). This is because hydrophilic and lipophilic ingredients can dissolve in the polar phase or the non-polar phase, respectively, and are therefore absorbed particularly quickly and to a high degree after application. The technology for combining lipophilic and hydrophilic substances in a carrier system can also be referred to as Microsolve+technology. Microsolve+technology is a further development of Microsolve technology, in which one or more lipophilic substances are finely distributed in the aqueous phase and virtually pre-dissolved (e.g., as micellization) using natural, food-typical solubilizers (e.g., soy lecithin). This facilitates the dispersion of the lipophilic nutrient in the gastrointestinal tract and thus supports optimal nutrient absorption.

Efficient absorption of the ingredients is also supported by ginger extract (Zingiber officinale), which acts as a bioenhancer to improve the absorption of ingredients by the human body. The presence of one or more bioenhancers enables the composition according to the invention to implement a nutrient transport concept (NTC). The lipophilic extract from ginger (Zingiber officinale) is preferably an extract from ginger rhizomes, preferably from dried ginger rhizomes. This may be ginger rhizome according to the European Pharmacopoeia. Preferably, the extract is a CO₂-extraction extract.

In the composition, the lipophilic (synonym: nonpolar) phase remains finely and homogeneously dispersed in the aqueous (synonym: polar) phase over a long period of time. Thus, the composition according to the invention is physically stable. Only lecithins can be used as emulsifiers, and these can be used in small quantities relative to the substance to be emulsified. For example, the mass of lecithins can be only 5% of the mass of the lipid to be emulsified. A further advantage is that lecithins are of natural origin.

Monohydric alcohols such as ethanol or propanol are not necessary for the stability of the composition. At the same time, however, their use in embodiments of the composition according to the invention is not excluded. The same applies to synthetic surfactants. One advantage is that the composition according to the invention is suitable for children, alcoholics, pregnant women, etc. when monohydric alcohols such as ethanol or propanol are not used. Another advantage is that the composition according to the invention is also suitable for people who reject synthetic surfactants when synthetic surfactants are not used.

The polyol contributes to preservation and, apart from the ingredients mentioned, no further preservatives are required. This means that synthetic or allergenic preservatives can be dispensed with.

Furthermore, lipophilic ingredients that are sensitive to oxidation and hydrolysis are surprisingly stable despite the presence of water and hydrophilic ingredients. The fact that oxidation is avoided not only increases the amount of lipophilic ingredients available, but also prevents the formation of products that are harmful to health.

Only naturally occurring ingredients can be used to form a stable composition with polar and nonpolar phases. This makes the composition particularly well accepted physiologically and psychologically and especially well tolerated. The composition presents a low risk of intolerances such as allergies.

In certain embodiments, the two-phase system is an emulsion.

Lecithin is a preparation containing phosphatidylcholine as its main component. Phosphatidylcholines are 1,2-diacyl-sn-glycero-3-phosphocholine, phospholipids in which phosphoric acid is esterified with diacylglycerol and choline. According to the Additive Approval Regulation (ZZulV 1998), lecithin is an additive approved for use in food in general (with the exception of certain foods) under the number E 322.

The composition according to the invention contains 0.5 g to 10 g, preferably 1 g to 3.5 g, of lecithin per 100 g of the composition. The lecithin is predominantly present in the boundary region between the polar and nonpolar phases. In other words, in addition to the polar phase and the nonpolar phase, the composition also comprises a lecithin phase consisting of the lecithin in the composition. The lecithin has a phosphatidylcholine content of 30 wt % to 100 wt %. In other words, the composition contains 0.15 g to 10 g, preferably 0.3 g to 3.5 g, of phosphatidylcholine per 100 g of the composition. In one embodiment, the composition according to the invention contains 1 g to 3 g of phosphatidylcholine per 100 g of the composition. Deoiled lecithin fractions are preferably used as lecithin. This means that the lecithin is preferably essentially free of nonpolar lipids such as fatty acids and triglycerides. The content of nonpolar lipids such as fatty acids and triglycerides is preferably less than 3% by weight. The lecithin contains a proportion of polar lipids (acetone-insoluble substance) of 90 to 100% by weight. The lecithin may be selected from a source of soybean, sunflower, rapeseed, fish, milk, eggs, and mixtures thereof, with non-animal sources such as soybean, sunflower, and rapeseed being particularly preferred. In addition to the main component phosphatidylcholine, lecithin may contain phosphatidylethanolamine, phosphatidylinositol, phosphatidylserine, sterols, fats, and oils, depending on its origin. For example, lecithin from soybeans may contain 40-50% by weight of phosphatidylcholine, approximately 10% by weight of phosphatidylethanolamine, approximately 5% by weight of phosphatidylinositol, approximately 1-2% by weight of phosphatidylserine, and sterols, fats, and oils. Lecithin obtained from egg yolks, on the other hand, consists mainly of phosphatidylcholine.

Water and glycerin are components of the polar phase. The nonpolar phase can consist of fatty acids and their esters, in particular triglycerides (triacylglycerols) and ethyl esters, fat-soluble vitamins, and carotenoids such as lutein and zeaxanthin, vitamin A (retinol) and its derivatives retinal, retinyl palmitate, and retinyl acetate, vitamin E as a collective term for tocopherols and tocotrienols (including alpha-, beta-, gamma-, and delta-tocopherols and tocotrienols, both as natural mixtures and synthetically in pure form), vitamin E derivatives such as alpha-, beta-, gamma-, and delta-tocopherol acetate (both in optically pure form and as race-mates), vitamin D2 (ergocalciferol), vitamin D3 (cholecalciferol), vitamin K1 (phylloquinone), vitamin K2 (menaquinone), vitamin K3 (menadione), coenzyme Q 10 H (ubiquinol), ubiquinone-10, and perfluorocarbons. In the present invention, the extract or extracts may also be used as a source for the components of the nonpolar phase.

When calculating the proportion of ingredients in the polar phase relative to the total weight of the polar phase, lecithin is not considered a component of the polar phase. When calculating the weight percentage, in case of doubt, those lipophilic compounds that contain a phosphate/phosphoric acid residue are considered lecithin and not a lipophilic ingredient, unless otherwise specified in the description. Lipophilic substances that do not contain a phosphate/phosphoric acid residue are considered lipophilic ingredients in case of doubt, unless otherwise specified in the description.

Embodiments within this document may be combined with each other as desired, unless the subject matter and description of the embodiments clearly indicate otherwise.

The verbs “contain” and “comprise” and their conjugations also include the verb “consist of” and its conjugations.

Preferred embodiments of the invention are specified in the subclaims.

In a preferred embodiment, the composition is a liquid at 20° C. Preferably, the liquid has a viscosity between 100 mPa·s and 3000 mPa·s at 20° C. The liquid dosage form allows for various applications and increases the freedom of movement of the ingredients within the composition, which can improve the absorption of the ingredients by the human body. Due to its liquid form, the composition according to the invention can implement a nutrient transport concept (NTC). This is because the liquid formulation can enable particularly rapid and efficient absorption of the ingredients.

The viscosity mentioned herein is the dynamic viscosity, measured according to the method of the European Pharmacopoeia 7.0, 2011, using a cone-plate rotary viscometer.

The composition preferably contains liposomes. Liposomes can be formed in particular when the composition contains more lecithin/phosphatidylcholine than is necessary for emulsification of the nonpolar phase. The emulsifier molecules (lecithin) that are not required to stabilize the nonpolar phase (e.g., fat droplets) can form liposomal structures in the polar phase. These can serve as a special carrier system for the water-soluble, hydrophilic active ingredients, so that two special carrier systems are present, namely fat droplets for lipophilic active ingredients and liposomes for hydrophilic active ingredients. The formation of liposomes can be supported by using high-pressure homogenizers in the manufacture of the composition. As mentioned at the beginning, the technology for combining lipophilic and hydrophilic substances in a carrier system can also be referred to as Microsolve⁺technology. Hydrophilic and lipophilic ingredients can dissolve in the polar phase or the non-polar phase, respectively, and thus be absorbed particularly quickly and to a high degree after application, enabling a nutrient transport concept (NTC) to be realized.

Preferably, the polyol is one or more compounds selected from glycerin, erythritol, sorbitol, and fructose. These are particularly suitable for stabilizing the composition. Glycerin is particularly preferred as a polyol.

Preferably, the polar phase contains 40 wt % to 96 wt %, preferably 60 wt % to 90 wt %, of polyol based on the total weight of the polar phase. A corresponding polyol content in the polar phase ensures sufficient preservation of the composition.

The polar phase contains at least 3 wt % and less than 20 wt % water based on the total weight of the polar phase. Preferably, the polar phase contains 5 wt % to 17 wt % water based on the total weight of the polar phase.

In a preferred embodiment, the composition contains lutein and zeaxanthin as further lipophilic ingredients.

In a preferred embodiment, the composition contains 0.01 g to 4.0 g of an extract from mint, preferably peppermint (Mentha piperita), per 100 g of the composition as a further lipophilic ingredient. Surprisingly, the mint extract further improves the absorption of the ingredients by the human body. The mint extract thus acts as a bioenhancer. Due to the presence of one or more bioenhancers, the composition according to the invention can implement a nutrient transport concept (NTC). The extract from mint or peppermint is preferably an extract from the above-ground parts, preferably the flowering above-ground parts. This may be peppermint oil in accordance with the European Pharmacopoeia. The extract may also be obtained by steam extraction. Preferably, it is a CO₂-extraction extract.

In a preferred embodiment, the composition contains at least one omega-3 fatty acid selected from docosahexaenoic acid, alpha-linolenic acid, and eicosapentaenoic acid as a further lipophilic ingredient. Docosahexaenoic acid (DHA) and eicosapentaenoic acid (EPA) form an important part of the human diet. Often, dietary habits or options do not meet the need for omega-3 fatty acids. Available omega-3 supplements, especially in the form of fish oil capsules, are often found to be indigestible, cause unwanted belching, and have an unpleasant fishy smell and taste. Capsules are also rejected due to fear of prions, a vegetarian or vegan lifestyle, or religious reasons. The direct intake of oily substances in general, and omega-3 fatty acid-containing oils in particular, is also often perceived as unpleasant and/or has negative connotations, leading to poor compliance and a continued lack of omega-3 fatty acid intake. The composition according to the invention masks the taste and smell of these components and thus improves compliance. The composition according to the invention also supports the absorption of these compounds by the human body and, surprisingly, allows the oxidation-and hydrolysis-sensitive omega-3 fatty acids or esters to remain stable despite the presence of water, hydrophilic ingredients, and oxygen. The Nutrient Transport Concept (NTC) and Microsolve technology can enable particularly high cellular absorption of poly-unsaturated fatty acids.

In a preferred embodiment, the composition contains coenzyme Q10 as a further lipophilic ingredient. The composition according to the invention supports the stability of this compound and its absorption by the human body. The Nutrient Transport Concept (NTC) and Microsolve technology can enable particularly high bioavailability of coenzyme Q10.

Preferably, the at least one hydrophilic ingredient is selected from vitamin C, vitamin B1, vitamin B2, vitamin B3, vitamin B6, vitamin B7(biotin), vitamin B12, minerals, trace elements, and oligomeric proanthocyanidins.

In a preferred embodiment, the composition contains an isoflavone.

Preferably, the composition contains 0.01 g to 2.0 g of the lipophilic extract from ginger per 100 g of the composition. It can best exert its effect in this quantity range. Due to the presence of one or more bioenhancers, the composition according to the invention can implement a nutrient transport concept (NTC).

In a preferred embodiment, the composition contains, as a further lipophilic ingredient, 0.5 g to 5.0 g of triacylglycerols, preferably medium-chain triacylglycerols, per 100 g of the composition. Triacylglycerols can serve as carriers for lipophilic ingredients. Their use can stabilize the formulation. Furthermore, the triacylglycerols form an oil film that adheres to the body part over a longer period of time, through which lipophilic ingredients can diffuse and take effect.

In a preferred embodiment, the composition contains at least one lipophilic ingredient selected from vitamin D2, vitamin D3, derivatives of vitamin D, vitamin A and derivatives thereof, vitamin E and derivatives thereof, vitamin K1, vitamin K2, isoprenoids, curcumin, berry extract, hawthorn extract, and artichoke extract. Curcumin in particular can further improve the availability of the ingredients to the human body, which is why curcumin is a preferred lipophilic ingredient. The Nutrient Transport Concept (NTC) and Microsolve technology can enable particularly high bioavailability of, for example, the applied vitamin E. The Nutrient Transport Concept (NTC) and Microsolve technology can also enable particularly high cellular uptake of phytosterols, for example. In addition, the composition according to the invention can implement a Nutrient Transport Concept (NTC) through the presence of one or more bioenhancers.

In preferred embodiments, the mass ratio of lecithin to lipophilic ingredient or to the lipophilic ingredients in the composition is between 8:1 and 1:20, particularly preferably between 1:1 and 1:12.

The composition preferably does not contain any ingredients of animal origin. In other words, the composition is suitable for a vegetarian and/or vegan lifestyle, in particular a vegetarian and/or vegan diet. Vegetarians and especially vegans often suffer from a deficiency of certain compounds and corresponding deficiency symptoms due to their limited food supply. It is advantageous that these people can be offered another product to ensure a more balanced diet.

Preferably, the composition does not contain any synthetic surfactant. The term “none” is to be understood as meaning that the concentration of the compound is so low that no significant effect is to be expected. In other words, the concentration is outside the range in which the compound is normally used. Synthetic surfactants within the meaning of the invention are ethoxylated surfactants, macrogol and derivatives thereof (e.g., macrogol glycerol fatty acid esters such as macrogol glycerol ricinoleate, macrogol fatty acid esters such as macrogol oleate, macrogol fatty alcohol ethers such as polyoxyethylene cetyl ether), propylene glycol and derivatives thereof, polyoxypropylene-polyoxyethylene block polymers, cellulose and derivatives thereof (e.g., hypromellose), benzalkonium, benzethonium, cetylalkonium, cetylpyridinium, cetyltrimethylammonium, dequalinium, sodium dodecyl sulfate, sodium cetyl sulfate, sodium stearyl sulfate, ethylene glycol monostearate, partial fatty acid esters of sorbitan (e.g., sorbitan monolaurate, sorbitan monopalmitate, sorbitan monostearate, sorbitan tristearate, sorbitan monooleate, sorbitan sesqui-oleate, sorbitan trioleate, polysorbates (polyoxyethylene sorbitan fatty acid esters), sucrose fatty acid esters, polyglycerol fatty acid esters, and D-α-tocopheryl 1000 succinate.

Preferably, the composition does not contain any artificial preservatives. The term “none” is to be understood as meaning that the concentration of the compound is so low that no significant effect is to be expected. In other words, the concentration is outside the range in which the compound is normally used. Artificial preservatives within the meaning of the invention are 4-hydroxybenzoic acid esters (e.g., 4-hydroxybenzoic acid methyl ester and 4-hydroxybenzoic acid propyl ester), chlorocresol, chlorobutanol, sorbic acid (E 200), potassium sorbate (E 202), calcium sorbate (E 203), sodium edetate, glycerol triacetate, benzalkonium, benzethonium, cetylalkonium, cetylpyridinium, c, cetyltrimethylammonium, cetrimonium, chlorhexi-dine, dequalinium, and organic mercury compounds.

Preferably, the composition does not contain any artificial sweeteners. The term “none” is to be understood as meaning that the concentration of the compound is so low that no significant effect is to be expected. In other words, the concentration is outside the range in which the compound is normally used. Artificial sweeteners within the meaning of the invention are acesulfame (E 950), advantame (E 969), aspartame (E 951), aspartame-acesulfame salt (E 962), cyclamate (E 952), neohe-speridine (E 959), neotame (E 961), saccharin (E 954), and sucralose (E 955) are considered artificial sweeteners within the meaning of the invention. Steviol glycosides, e.g., stevioside (E 960), are not considered artificial sweeteners within the meaning of the invention.

In a preferred embodiment, the composition does not contain any monohydric alcohol. In a preferred embodiment, the composition contains a maximum of 1 g of monohydric alcohols per 100 g of the composition. Monohydric alcohols within the meaning of this document include, for example, ethanol and propanol.

In a preferred embodiment, the composition does not contain any dihydric alcohol. In a preferred embodiment, the composition contains a maximum of 1 g of dihydric alcohols per 100 g of the composition. Propylene glycol, for example, is considered a dihydric alcohol within the meaning of this document.

A drip device comprising a composition according to the invention is also claimed, wherein the composition according to the invention is a liquid.

The invention is also based on the idea of specifying a method for producing a composition according to the invention.

The method for producing a composition according to the invention preferably comprises the steps of:
a) stirring an aqueous solution, wherein the aqueous solution contains lecithin and at least 20% by weight of polyol, at least 3% by weight and less than 20% by weight of water, and at least one hydrophilic ingredient,
b) adding a lipophilic extract from ginger (Zingiber officinale) and at least one other lipophilic ingredient to obtain a mixture, wherein the mixture contains 0.5 g to 10 g of lecithin per 100 g,
c) stirring the mixture and homogenizing it under high pressure to obtain the composition according to the invention.

The process temperature is preferably adapted to the lecithin and the lipophilic ingredient to be solubilized. For hydrogenated lecithins, processing temperatures between 40° C. and 80° C. are preferred; for unsaturated lecithins, room temperature can be used. It is preferable to work above the melting point of lipophilic ingredients.

Homogenization is preferably carried out using a high-pressure homogenizer, ultrasound, and/or a rotor-stator mixer, in particular a high-pressure homogenizer. A high-pressure homogenizer produces more transparent two-phase systems, as the inner phase is dispersed more finely. A high-pressure homogenizer ensures that the composition contains liposomes. Surprisingly, the degree of autoxidation and hydrolysis of the oils, fats, or lipids in the composition according to the invention is low, even though air and water are incorporated using a high-pressure homogenizer.

The process, in particular stirring and homogenization under high pressure, is preferably carried out under inert gas.

For further embodiments, advantages, and explanations of the process, reference is made to the descriptions of the composition.

The invention is also based on the idea of specifying the use of a composition according to the invention as a dietary supplement or cosmetic preparation. Use as a dietary supplement is particularly preferred.

The composition is particularly suitable for use as a dietary supplement and/or cosmetic preparation, as it overcomes the disadvantages of conventional products. The composition enables the simultaneous provision and good absorption of hydrophilic and lipophilic ingredients by the human body. Only by using naturally occurring ingredients and without the use of monohydric or dihydric alcohols, synthetic surfactants, or preservatives is it possible to create a physically, chemically, and microbiologically stable composition that is particularly effective at facilitating the absorption of ingredients by the human body. The composition is particularly well accepted physiologically and psychologically and is especially well tolerated. The taste and smell are not perceived as unpleasant, but as very good. When taken orally, belching is prevented. Since no (gelatin) capsules are used, the preparations according to the invention are also available to people who reject capsules due to fear of prions, because of a vegetarian or vegan lifestyle, or for religious reasons. The two-phase system has an advantageous viscosity.

The preferred use is topical or oral, with oral use being particularly preferred.

For further embodiments, advantages, and explanations, reference is made to the descriptions of the composition.

The invention is explained in more detail below with reference to embodiments.

### Example 1

2.5 g of soy lecithin with a phosphatidylcholine content of 45% and 0.2 g of vitamin B₂ are completely dispersed in 92 g of an 85% (wt %) aqueous glycerol solution while stirring at 50° C.

0.3 g of a lipophilic extract from ginger and 5 g of vitamin E acetate are now added to this dispersion and stirred homogeneously at 50° C. Finally, homogenization is carried out using a high-pressure homogenizer.

The result is a stable dispersion whose components can be absorbed particularly well by the human body.

### Example 2

2 g of sunflower lecithin with a phosphatidylcholine content of 75% and 0.5 g of niacinamide are completely dispersed in 85.5 g of an 85% (wt %) aqueous glycerol solution while stirring at 50° C.

0.5 g of a lipophilic extract from ginger, 2 g of coenzyme Q10, and 10 g of algae oil are now added to this dispersion and stirred homogeneously at 50° C. Finally, homogenization is carried out using a high-pressure homogenizer.

The result is a stable dispersion whose components can be absorbed particularly well by the human body.

1. Composition, wherein the composition comprises a polar phase and a non-polar phase, wherein
the composition contains a lipophilic extract from ginger (Zingiber officinale) and at least one other lipophilic ingredient,
the polar phase contains at least 20% by weight of polyol, at least 3% by weight and less than 20% by weight of water, and at least one hydrophilic ingredient, based on the total weight of the polar phase, and
the composition contains 0.5 g to 10 g of lecithin per 100 g of the composition.

2. Composition according to claim 1, characterized in that the composition has a viscosity between 100 mPa·s and 3000 mPa·s at 20° C.

3. Composition according to claim 1, characterized in that the composition contains an isoflavone.

4. Composition according to claim 1, characterized in that the composition contains liposomes.

5. Composition according to claim 1, characterized in that the polar phase contains 40 wt % to 96 wt %, preferably 60 wt % to 90 wt %, of polyol based on the total weight of the polar phase.

6. Composition according to claim 1, characterized in that the polyol is glycerin.

7. Composition according to claim 1, characterized in that the composition contains lutein and zeaxanthin.

8. Composition according to claim 1, characterized in that the composition contains 0.01 g to 4.0 g of an extract from mint, preferably from peppermint (Mentha piperita), per 100 g of the composition.

9. Composition according to claim 1, characterized in that the composition contains an omega-3 fatty acid selected from docosahexaenoic acid, alpha-linolenic acid, and eicosapentaenoic acid.

10. Composition according to claim 1 to 9, characterized in that the composition contains coenzyme Q10.

11. Composition according to claim 1, characterized in that the composition contains, as a hydrophilic ingredient, at least one ingredient selected from vitamin C, vitamin B1, vitamin B2, vitamin B3, vitamin B6, vitamin B7(biotin), vitamin B12, minerals, trace elements, and oligomeric proanthocyanidins as a hydrophilic ingredient.

12. Composition according to claim 1 to 11, characterized in that the composition contains 0.01 g to 2.0 g of the lipophilic extract from ginger per 100 g of the composition.

13. Composition according to claim 1 to 12, characterized in that the composition contains 0.5 g to 5.0 g of triacylglycerols, preferably medium-chain triacylglycerols, per 100 g of the composition.

14. Composition according to claim 1, characterized in that the composition contains at least one lipophilic ingredient selected from vitamin D2, vitamin D3, derivatives of vitamin D, vitamin A and derivatives thereof, vitamin E and derivatives thereof, vitamin K1, vitamin K2, isoprenoids, curcumin, berry extract, hawthorn extract, and artichoke extract.

15. Composition according to claim 1, characterized in that the composition contains curcumin.
